(19) United States
(12) Patent Application Publication
(10) Pub. No.: US 2018/0303747 A1
(43) Pub. Date: Oct. 25, 2018

(54) SCALP RELIEVING MIXTURE AND SPRAY

(71) Applicant: Charlotte Higginbotham - Van Horn, Woodbridge, VA (US)

(72) Inventor: Charlotte Higginbotham - Van Horn, Woodbridge, VA (US)

(21) Appl. No.: 15/956,496

(22) Filed: Apr. 18, 2018

Related U.S. Application Data
(60) Provisional application No. 62/487,559, filed on Apr. 20, 2017.

(51) Int. Cl. A61K 8/92; A61K 8/9794; A61K 8/04; A61Q 5/04

(52) U.S. Cl. CPC A61K 8/922; A61Q 5/04; A61K 8/046; A61K 8/9794

(57) ABSTRACT

A scalp relieving mixture and spray to alleviate pain on an individual's scalp associated with managing, manipulating, and styling hair on the individual's scalp. The scalp relieving mixture and spray includes pure essential oils from the group consisting of clove bud oil, lavender oil, lemongrass oil, peppermint oil, and tea tree oil, Aloe Vera gel, and distilled water.

### CROSS-REFERENCE TO RELATED APPLICATIONS

This application claims the benefit of U.S. Provisional Patent Application No. 62/487,559 filed on Apr. 20, 2017 which is incorporated in its entirety herein by reference.

### TECHNICAL FIELD OF THE INVENTION

The present invention relates to a scalp relieving mixture and spray. More specifically, the invention is directed to a scalp relieving mixture and spray directly applied to an individual's scalp to alleviate pain.

### BACKGROUND OF THE INVENTION

Hair is a filamentous biomaterial that grows from follicles found in the dermis. Human hair has many textures, from fine to coarse, and from straight to curly. Hair generally can be divided into four categories: (i) straight, (ii) wavy, (iii) curly, and (iv) kinky. The human hair shaft is comprised of three concentric layers identified as the cuticle (a thin, outer-most shell); the cortex (the main body of the hair); and the medulla (a thin, central core). The cuticle and cortex are responsible for the hair shaft's mechanical properties, that is, its tendency to curl (in some hair the medulla is absent). The condition of the cuticle is responsible for the outward appearance of the hair, particularly feel and shine. Straight hair resembles a rod with a circular diameter; wavy hair shafts are compressed into an oval diameter; curly shafts are further compressed into an elongated ellipse; and kinky hair shafts are flatter still.

Many people with naturally kinky, curly, or even wavy hair often desire to straighten their hair. There are a number of hair straightening compositions and methods available today, but these involve use of harsh straightening agents such as alkaline or sulfite-based chemicals. Most permanent straightening products marketed today use one of five types of chemicals: (1) thioglycolate-based products, (2) sulfite-bisulfite products, (3) sodium hydroxide (alkali) products, (4) lithium hydroxide, and (5) acetamide (formulated as a between-shampoos, aerosol styling aid). In all these cases excepting acetamide, the chemical agent is applied to clean, damp hair that has been pulled straight by combing action. After a carefully-measured period of time, the disulfide bonds are broken, the solution is rinsed off, and a second solution (often hydrogen peroxide or other oxidizing agent) is applied to neutralize the reaction.

### SUMMARY OF THE INVENTION

According to an embodiment of the present invention, there is disclosed a scalp relieving mixture and spray to alleviate pain on an individual's scalp associated with managing, manipulating, and styling hair on the individual's scalp. The scalp relieving mixture and spray includes pure essential oils from the group consisting of clove bud oil, lavender oil, lemongrass oil, peppermint oil, and tea tree oil, Aloe Vera gel, and distilled water.

According to another embodiment of the present invention, there is disclosed a method of relieving pain on an individual's scalp associated with managing, manipulating, and styling the hair on the individual's scalp. The method includes consisting mixing a mixture of pure essential oils from the group consisting of clove bud oil, lavender oil, lemongrass oil, peppermint oil, and tea tree oil with Aloe Vera gel and distilled water. Then, the method includes washing the hair of the individual with shampoo to obtain washed hair, and drying the individual's washed hair to obtain dried hair. Then, spraying the mixture of pure essential oils from the group consisting of clove bud oil, lavender oil, lemongrass oil, peppermint oil, and tea tree oil with Aloe Vera gel and distilled water onto the individual's scalp. Finally, the method includes achieving a desired hair style utilizing a hair styling formulation.

### BRIEF DESCRIPTION OF THE DRAWINGS

In the description that follows, numerous details are set forth in order to provide a thorough understanding of the present invention. It will be appreciated by those skilled in the art that variations of these specific details are possible while still achieving the results of the present invention. Well-known processing steps are generally not described in detail in order to avoid unnecessarily obfuscating the description of the present invention.

In the description that follows, exemplary dimensions may be presented for an illustrative embodiment of the invention. The dimensions should not be interpreted as limiting. They are included to provide a sense of proportion. Generally speaking, it is the relationship between various elements, where they are located, their contrasting compositions, and sometimes their relative sizes that is of significance.

In the drawings accompanying the description that follows, often both reference numerals and legends (labels, text descriptions) will be used to identify elements. If legends are provided, they are intended merely as an aid to the reader, and should not in any way be interpreted as limiting.

FIG. 1 is a front view of the scalp relieving mixture and spray in use, in accordance with the present invention.

FIG. 2 is a front view of the method of using the scalp relieving mixture and spray, in accordance with the present invention.

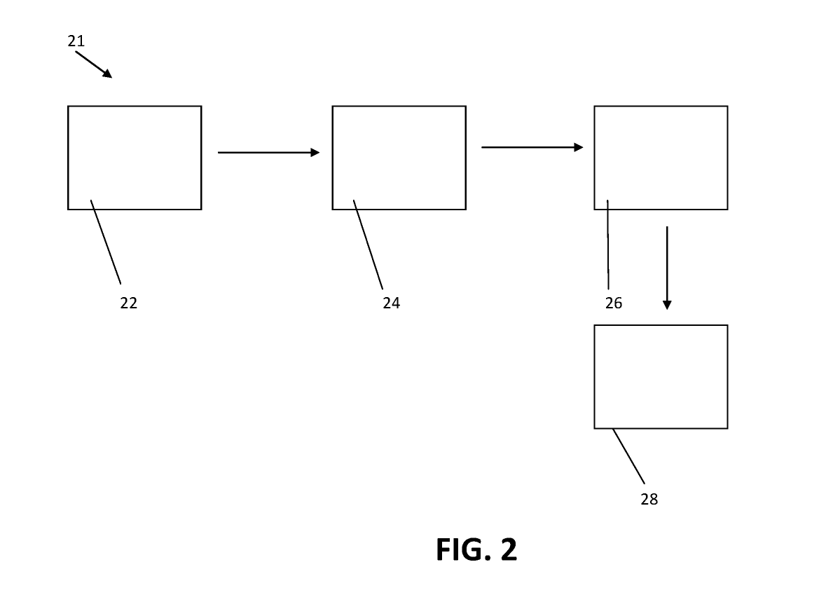

### DETAILED DESCRIPTION OF THE PREFERRED EMBODIMENTS

In the description that follows, numerous details are set forth in order to provide a thorough understanding of the present invention. It will be appreciated by those skilled in the art that variations of these specific details are possible while still achieving the results of the present invention. Well-known processing steps are generally not described in detail in order to avoid unnecessarily obfuscating the description of the present invention.

In the description that follows, exemplary dimensions may be presented for an illustrative embodiment of the invention. The dimensions should not be interpreted as limiting. They are included to provide a sense of proportion. Generally speaking, it is the relationship between various elements, where they are located, their contrasting compositions, and sometimes their relative sizes that is of significance.

In the drawings accompanying the description that follows, often both reference numerals and legends (labels, text descriptions) will be used to identify elements. If legends are provided, they are intended merely as an aid to the reader, and should not in any way be interpreted as limiting.

Curly hair is made of hair strands that have irregular surfaces that mesh and tangle with each other to make combing more difficult. Among individuals with very curly hair, e.g., individuals of African or Middle Eastern descent, it is especially popular to relax or straighten hair to increase hair manageability and ease of styling.

Conventional systems for treating or beautifying the hair or scalp generally rely on applying liquid products via washing-and-rinse-off or spraying regimes. A common complaint by people having their hair straightened, relaxed or otherwise styled is that the process can be quite painful, especially to the scalp.

The scalp relieving mixture and spray 10 is a product designed to be applied to an individual's scalp. By applying the scalp relieving mixture and spray 10 directly to the scalp, pain associated with the hair is alleviated. For example, hair straightening is a hair styling technique which involves the flattening and straightening of hair in order to give it a smooth, streamlined, and ‘sleek’ appearance. It may be accomplished using a hair iron or hot comb, chemical relaxers, Japanese hair straightening, or Brazilian hair straightening. In addition, some shampoos, and conditioners and hair gels can help to make hair temporarily straight.

Hair irons and hot combs can only temporarily modify hair texture, whereas relaxers and the other methods permanently alter the structure of the hair, although new hair growth is not affected. In any type of hair styling, where there is a possibility that the person's scalp will be tender and painful, the scalp relieving mixture and spray 10 can be applied directly to the scalp whereby pain associated with the hair is alleviated or at least reduced.

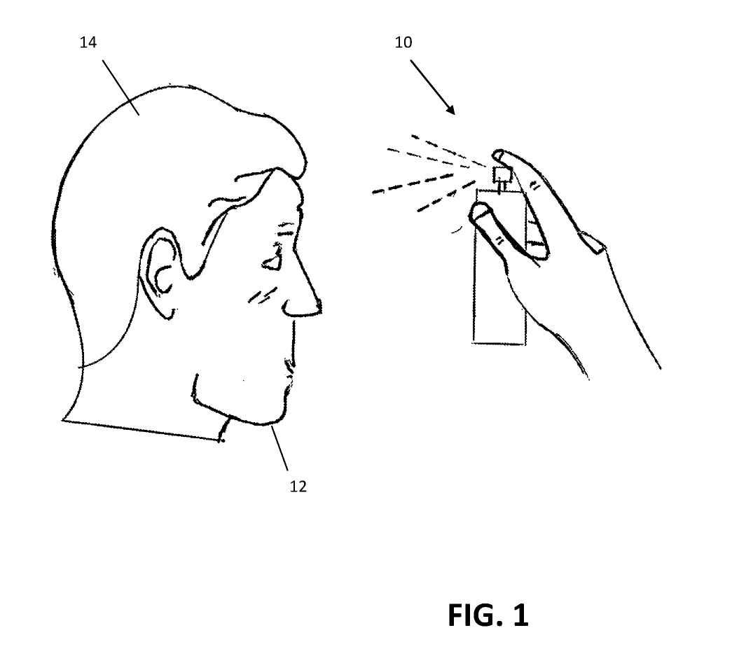

As seen in FIG. 1, the scalp relieving mixture and spray 10 is designed to alleviate the painful issues associated with managing, manipulating, and/or styling the hair on an individual's 12 scalp 14. The scalp relieving mixture and spray 10 includes a combination of pure essential oils, pure Aloe Vera gel and distilled water that are applied to the hair 14 of an individual 12. When the scalp relieving mixture and spray 10 is sprayed on to the scalp 14, the individual experiences relief from discomfort during styling, through heat and relaxation benefits of ingredients.

The scalp relieving mixture and spray 10 is designed to deliver the specific benefit agents directly to the hair 14 and/or scalp. The ingredients of the scalp relieving mixture and spray 10 include clove bud, lavender, lemongrass, peppermint, tea tree oil, pure aloe vera gel and purified water, poured into a mist spray bottle. It is within the scope of the present invention to include other desired ingredients.

The particular ingredients act in harmony with each other to achieve the desired results of the scalp relieving mixture and spray 10. It has been determined that the general combination of these particular ingredients result in a desired potency and pleasant aroma quality. The scalp relieving mixture and spray 10 is preferably applied at room temperature.

The clove bud, lavender and lemon grass act to diminish pain and operate as a natural anesthetic and include anti-inflammatory benefits. The peppermint and tea tree increase heat and stimulate circulation. The aloe vera gel cools and moisturizes the skin surface of the scalp 14. Finally, the lavender encourages relaxation.

The combined ingredients of the scalp relieving mixture and spray 10 may be housed within a conventional spray bottle. Typically, spray bottles include a head and a bottle. The head includes a trigger member that activates a small pump inside the head. The pump is attached to a tube that draws fluid from the bottom of the bottle. In operation, the pump forces fluid drawn by the tube through a barrel and out a small hole or nozzle.

In another preferred embodiment, the combined ingredients of the scalp relieving mixture and spray 10 may be housed within a conventional aerosol can. In this embodiment, the ingredients are dispensed as an aerosol mist of liquid particles. The aerosol can that contains the scalp relieving mixture and spray 10 incorporates a valve. When opened, the ingredients are forced out of a small hole and emerge as an aerosol mist. As propellant expands to drive out the aerosol mist, only some propellant evaporates inside the can to maintain a constant pressure. Outside the can, the droplets of propellant evaporate rapidly, leaving the scalp relieving mixture and spray 10 suspended as very fine particles or droplets.

An exemplary method 21 of using the scalp relieving mixture and spray 10 may include the following steps. First, in step 22, the hair of the individual is washed with shampoo to obtain washed hair. The shampoo will swell and open the cuticle and make it semi-porous due to the breakage of the disulfide bonds.

Then, in step 24, the method provides for drying the individual's washed hair of step 22 to obtain dried hair. This may be accomplished with a hair dryer, a towel, or any other desired method.

In step 26, the scalp relieving mixture and spray is sprayed onto the individual's scalp, thereby diminishing pain, operating as a natural anesthetic, including anti-inflammatory benefits, increasing heat and stimulating circulation, and cooling and moisturizing the skin surface of the scalp 14.

In step 28, any desired hair styling formulation may be utilized to achieve the desired hair style. For example, a hair fixative may be applied that contains keratin and epigallocatechin gallate along with other ingredients in about one inch sections of hair, about 0.25 inches from scalp, using a brush and comb.

Although the invention has been shown and described with respect to a certain preferred embodiment or embodiments, certain equivalent alterations and modifications will occur to others skilled in the art upon the reading and understanding of this specification and the annexed drawings. In particular regard to the various functions performed by the above described components (assemblies, devices, etc.) the terms (including a reference to a “means”) used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments of the invention. In addition, while a particular feature of the invention may have been disclosed with respect to only one of several embodiments, such feature may be combined with one or more features of the other embodiments as may be desired and advantageous for any given or particular application.

1. A scalp relieving mixture and spray to alleviate pain on an individual's scalp associated with managing, manipulating, and styling hair on the individual's scalp, consisting of;
pure essential oils from the group consisting of clove bud oil, lavender oil, lemongrass oil, peppermint oil, and tea tree oil;
Aloe Vera gel; and
distilled water.

2. The scalp relieving mixture and spray of claim 1, wherein the combination of clove bud oil, the lavender oil and lemon grass oil alleviate pain associated managing, manipulating, and styling hair when applied to the individual's scalp.

3. The scalp relieving mixture and spray of claim 2, further wherein the combination of clove bud oil, the lavender oil and lemon grass oil operate as a natural anesthetic with anti-inflammatory benefits when applied to the individual's scalp.

4. The scalp relieving mixture and spray of claim 1, further wherein the combination of peppermint oil and tea tree oil increase heat and stimulating circulation when applied to the individual's scalp.

5. The scalp relieving mixture and spray of claim 1, further wherein the aloe vera gel cools and moisturizes skin surface of the scalp.

6. The scalp relieving mixture and spray of claim 1, further wherein the lavender oil aids in relaxation of the individual's scalp and hair.

7. The scalp relieving mixture and spray of claim 1, further including housing the pure essential oils from the group consisting of clove bud oil, lavender oil, lemongrass oil, peppermint oil, and tea tree oil; the Aloe Vera gel, and the distilled water within a conventional spray bottle.

8. The scalp relieving mixture and spray of claim 7, further including the conventional spray bottle having a head and a bottle.

9. The scalp relieving mixture and spray of claim 8, further including the head including a trigger member that activates a small pump inside the head, whereby the pump is attached to a tube that draws fluid from a bottom of the bottle.

10. The scalp relieving mixture and spray of claim 1, further including housing the pure essential oils from the group consisting of clove bud oil, lavender oil, lemongrass oil, peppermint oil, tea tree oil, the Aloe Vera gel, and the distilled water within a aerosol can, whereby the pure essential oils, the Aloe Vera gel, and the distilled water are dispensed as an aerosol mist of liquid particles.

11. The scalp relieving mixture and spray of claim 10, further including the aerosol can incorporating a valve, through which liquid particles of the clove bud oil, the lavender oil, the lemongrass oil, the peppermint oil, the tea tree oil, the Aloe Vera gel, and the distilled water are forced out of a small hole and emerge as an aerosol mist.

12. The scalp relieving mixture and spray of claim 11, further wherein the aerosol can contains a propellant which expands to drive out the aerosol mist through the small hole leaving some of the propellant inside the can to maintain a constant pressure and droplets of the propellant evaporating rapidly outside of the can leaving the scalp relieving mixture and spray suspended as very fine particles and droplets.

13. A method of relieving pain on an individual's scalp associated with managing, manipulating, and styling the hair on the individual's scalp, consisting of;
mixing a mixture of pure essential oils from the group consisting of clove bud oil, lavender oil, lemongrass oil, peppermint oil, and tea tree oil with Aloe Vera gel and distilled water;
washing the hair of the individual with shampoo to obtain washed hair;
drying the individual's washed hair to obtain dried hair;
spraying the mixture of pure essential oils from the group consisting of clove bud oil, lavender oil, lemongrass oil, peppermint oil, and tea tree oil with Aloe Vera gel and distilled water onto the individual's scalp; and
achieving a desired hair style utilizing a hair styling formulation.

14. The method of claim 13 further including drying the individual's washed hair with a hair dryer.

15. The method of claim 13 further including drying the individual's washed hair with a towel.

16. The method of claim 13 further including diminishing pain of the individual's scalp with the scalp relieving mixture and spray.

17. The method of claim 16 further including increasing heat and stimulating circulation of the individual's scalp.

18. The method of claim 17 further including cooling and moisturizing the skin surface of the individual's scalp.

19. The method of claim 13 further including achieving a desired hair style utilizing a hair fixative that contains keratin and epigallocatechin gallate.
